(19) United States
(12) Patent Application Publication
(10) Pub. No.: US 2010/0298509 A1
(43) Pub. Date: Nov. 25, 2010

(54) METHOD OF PRODUCING DIALKOXYMAGNESIUM SUPPORT FOR CATALYST FOR OLEFIN POLYMERIZATION, METHOD OF PRODUCING CATALYST FOR OLEFIN POLYMERIZATION USING THE SAME AND METHOD OF POLYMERIZING OLEFIN USING THE SAME

(75) Inventors: Eun II KIM, Daejon (KR); Jong Sik KIM, Daejon (KR); Young Joo LEE, Inchon (KR); Joon Ryeo PARK, Seoul (KR)

Correspondence Address: YOUNG & THOMPSON, 209 Madison Street, Suite 500, Alexandria, VA, 22314, US

(73) Assignee: SAMSUNG TOTAL PETROCHEMICALS CO., LTD., Seosan-shi (KR)

(21) Appl. No.: 12/686,734

(22) Filed: Jan. 13, 2010

(30) Foreign Application Priority Data
May 22, 2009 (KR) 10-2009-0044778

(51) Int. Cl. C07C 29/70; C08F 4/60; C08F 4/50

(52) U.S. Cl. 526/124.3; 502/104; 568/902

(57) ABSTRACT

Disclosed are a method for producing a dialkoxymagnesium support for catalyst for olefin polymerization, a method of producing catalyst for olefin polymerization using the dialkoxymagnesium support and a method of polymerizing olefin using the catalyst. By using the method for producing a support according to the present invention, the content of large particles in the dialkoxymagnesium support can be controlled and the particle can have spherical shape, so the catalyst produced by using the support have high activity and stereoregularity, and high bulk density, thereby making it possible to be applied to the commercial processes.

### TECHNICAL FIELD

The present invention relates to a method of producing a dialkoxymagnesium support for catalyst for olefin polymerization. The invention also relates to a method of producing catalyst for olefin polymerization using the dialkoxymagnesium support and a method of polymerizing olefin using the catalyst.

### BACKGROUND ART

The magnesium chloride-supported Ziegler-Natta catalyst is now most widely used as a catalyst for polymerizing olefin. The magnesium chloride-supported Ziegler-Natta catalyst generally consists of a solid catalyst component comprising magnesium, titanium, halogen and organic compounds of the type of electron donor, and, when used for polymerizing alpha-olefin such as propylene, is used mixed with a cocatalyst, organic aluminum, and a controller of stereoregularity, organic silane, with appropriate mixing ratio. Since the solid catalyst for olefin polymerization is used in various commercial procedures such as slurry polymerization, bulk polymerization and gas state polymerization, various requirements on the particle shape such as appropriate particle size and shape, homogeneous distribution of particle size, minimization of large and fine particles, and high bulk density should be met as well as the basic characteristics of high activity and stereoregularity.

Many methods for improving the particle morphology of the support for catalyst for olefin polymerization have been known in the field including methods such as recrystallization and reprecipitation method, spray dry method and methods using chemical reactions. Among these methods, the recrystallization and reprecipitation method has the problem that it is difficult to control the particle size in producing the support.

Recently, as one of the methods of using chemical reaction, a method of producing a catalyst by using a dialkoxymagnesium obtained by reacting magnesium with alcohol as a support is drawing attention due to its ability to control the size of the support as needed in the specific process or product as well as the ability to provide a catalyst having much higher activity and a polymer having high stereoregularity compared to other methods.

In the method which uses dialkoxymagnesium as a support, however, since the shape and distribution of size and bulk density of the dialkoxymagnesium particle directly affect the particle characteristics of the catalyst and polymer, it is necessary to prepare a dialkoxymagnesium support which has a uniform size, spherical shape and sufficiently high bulk density. Especially, lots of large particles of support can deteriorate the flowability of a polymer, making it difficult to apply the process to a production line.

Some methods of producing dialkoxymagnesium with uniform shape have been disclosed in prior arts. U.S. Pat. Nos. 5,162,277 and 5,955,396 disclose a method of producing a support with size of 5-10 μm by recrystallizing an amorphous magnesium methyl carbonate made by carboxylizing a diethoxymagnesium through CO₂ in a solution using various additives and solvent. Also Japanese Laid Open Patent 1994-87773 discloses a method of producing a spherical particle by spray drying and decarboxylizing an alcohol solution of diethoxymagnesium which has been carboxylized through CO₂. These conventional methods, however, require complicated processes using many kinds of materials and fail to provide a particle with satisfactory sizes and shapes.

Meanwhile, Japanese Laid Open Patents 1991-74341, 1992-368391 and 1996-73388 disclose a method of synthesizing a diethoxymagnesium in spherical or elliptical shape by reacting a magnesium metal with ethanol under the presence of iodine (I). These methods, however, have difficulty in controlling the reaction velocity properly since the reaction proceeds very rapidly with much reaction heat and lots of hydrogen, and the resulting dialkoxymagnusium support contains large amount of fine particles or multi-type large particles in which a number of particles are condensed. Also, when the catalyst which is produced from the above support is directly used in olefin polymerization process, there can be problem of excessively large particle size of the polymer, and destruction of particle shape by polymerization heat generated in the polymerization process, causing severe damage in the process.

### SUMMARY OF THE INVENTION

The present invention has been designed to solve the above mentioned problems of prior arts and, in order to produce a catalyst that can fulfill the particle characteristics requirement needed in the process of commercial olefin polymerization such as slurry polymerization, bulk polymerization and gas state polymerization, aims to provide a method of producing a dialkoxymagnesium support for the catalyst for olefin polymerization, which has uniform particle distributions and smooth surface, by minimizing the amount of large particles in the support. The invention also aims to provide a method of producing catalyst for olefin polymerization using the above-prepared support, and a method of polymerizing olefin using thus produced catalyst.

### DISCLOSURE

In order to achieve the above mentioned objective, the method of producing a dialkoxymagnesium support for catalyst for olefin polymerization according to the present invention comprises reacting a magnesium metal with an alcohol under the presence of an initiator, N-chlorosuccinimide, at the initial reaction temperature of 40-60° C.

While there is no specific limitation in the shape of the magnesium metal used in the method of producing a dialkoxymagnesium support, the magnesium metal is preferably in the form of powder with average particle size 10-300 μm, or more preferably, in the form of powder with average particle size 50-200 μm. When the average particle size of the magnesium metal is less than 10 μm, the average particle size of the resulting support becomes too fine, and when the average particle size is large than 300 μm, the average particle size of the support becomes too large and it is difficult to render the support in the shape of uniform sphere.

There is no specific limitation in the alcohol used in the method of producing a dialkoxymagnesium support, but it is preferable to use one or more of alcohol selected from the aliphatic alcohol represented by general formula of ROH (where R is C₁₋₆ alkyl group) such as methanol, ethanol, normal propanol, isopropanol, normal butanol, isobutanol, normal pentanol, isopentanol, neopentanol, cyclopentanol and cyclohexanol, or aromatic alcohol such as phenol, or more preferably, one or more of alcohol selected from methanol, ethanol, propanol and butanol, or most preferably, ethanol.

The amount of used alcohol is preferably 5-50 parts by weight per 1 part by weight of the magnesium metal, or more preferably, 7-20 parts by weight per 1 part by weight of the magnesium metal. When less than 5 parts by weight of alcohol is used, the viscosity of slurry rapidly increases, and when more than 50 parts by weight of alcohol is used, the bulk density of the produced support decreases and poses the problem of generating particles of rough surface.

In the method of producing a dialkoxymagnesium support, N-chlorosuccinimide is used as an initiator. The use of N-chlorosuccinimide as an initiator provides the merit of suppressing the generation of large particles compared to the use of conventional initiator such as N-bromosuccinimide.

The amount of N-chlorosuccinimide used as an initiator is preferably 0.001-0.2 parts by weight per 1 part by weight of the magnesium metal. When less than 0.001 parts by weight of N-chlorosuccinimide is used, the reaction velocity becomes too slowed, and when more than 0.2 parts by weight is used, there is a problem that the size of resultant particles becomes too large or too many fine particles are generated.

The process of producing the support is carried out by first reacting a magnesium metal with an alcohol under the presence of the initiator, and by performing aging at raised temperatures, with the initial reaction temperature of 40-60° C. and aging temperature of preferably 75-90° C. When the initial reaction temperature is lower than 40° C., the reaction is not easily started making the reaction time longer, and when the initial reaction temperature is higher than 60° C., it is difficult to obtain low content of large particles. Stirring is carried out preferably with the velocity of 50-300 rpm, or more preferably, with the velocity of 70-250 rpm. When the stirring velocity is outside the preferred range, there is the shortcoming of irregular particle distribution.

The method for producing a catalyst for olefin polymerization according to the present invention features in contact-reacting the dialkoxymagnesium support produced by the above mentioned method of the present invention with a titanium halide compound and an internal electron donor.

In the above production of a catalyst, a multiporous solid catalyst particle is obtained by first reacting a dialkoxymagnesium in the shape of uniform spherical particle with a titanium halide compound under the presence of organic solvent to substitute the alkoxy group of the dialkoxymagnesium with halogen group, and then by reacting the titanium halide compound and the internal electron donor under the presence of organic solvent at 0-130° C.

Although any type of titanium halide compound can be used for producing the catalyst, titanium tetrachloride is preferable.

The organic solvent used in the above production of a catalyst can be aliphatic hydrocarbon having 6-12 of carbon atoms or aromatic hydrocarbon, or preferably, saturated aliphatic hydrocarbon having 7-10 of carbon atoms or aromatic hydrocarbon specifically such as octane, nonane, decane, or toluene and xylene.

The internal electron donor used in the above production of a catalyst can be preferably diester, or more preferably aromatic diester, or most preferably phtalic acid diester. Examples of phtalic acid diester are one or more selected from dimethylphtalate, diethylphtalate, dinormalpropylphtalate, diisopropylphtalate, dinormalbutylphtalate, diisobutylphtalate, dinormalpentylphtalate, di(2-methylbutyl)phtalate, di(3-methylbutyl)phtalate, dineopentylphtalate, dinormalhexylphtalate, di(2-methylpentyl)phtalate, di(3-methylpentyl)phtalate, diisohexylphtalate, dineohexylphtalate, di(2,3-dimethylbutyl)phtalate, dinormalheptylphtalate, di(2-methylhexyl)phtalate, di(2-ethylpentyl)phtalate, diisoheptylphtalate, dineohepylphtalate, dinormaloctylphtalate, di(2-methylheptyl)phtalate, diisooctylphtalate, di(3-ethylhexyl)phtalate, dine ohexylphtalate, dinormalheptylphtalate, diisoheptylphtalate, dineoheptylphtalate, dinormaloctylphtalate, diisooctylphtalate, dineooctylphtalate, dinormalnonylphtalate, diisononylphtalate, dinormaldecylphtalate, diisodecylphtalate and the like, which are represented by the general formula below.

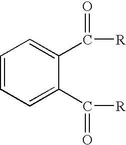

where R is an C₁₋₁₀ alkyl group.

In the above production of a catalyst, the contact and reaction of each component are carried out under an inert gas atmosphere in a reactor equipped with a stirrer with water being sufficiently removed. The contact of the dialkoxymagnesium support and the titanium halide compound is carried out in the state suspended in the aliphatic or aromatic solvent at 0-50° C., or more specifically at 10-30° C. Outside these contact temperatures, there can be a problem of generating lots of fine particles due to the destruction of the shape of the support particle. The amount of titanium halide compound used at this step is preferably 0.1-10 mol, or more preferably, 0.3-2 mole per 1 mole of the dialkoxymagnesium, and the titanium halide is injected preferably slowly over 30 minutes to 3 hours. After the injection is finished, the temperature is slowly raised to 40-80° C., thereby completing the reaction. After the reaction is completed, the mixture in slurry state is washed once or more with toluene. And then a titanium halide compound is injected and the temperature is raised to 90-130° C. for aging. The amount of the titanium halide used at this step is preferably 0.5-10 mole, or more preferably, 1-5 mole per 1 mole of the dialkoxymagnesium. While raising the temperature, an internal electron donor should be injected, and while the temperature or the number of injection of the electron donor is not strictly limited, the total amount of the internal electron donor used is preferably 0.1-1.0 parts by weight per 1 part by weight of the dialkoxymagnesium used. When the internal electron donor is used in the amount outside the preferred range, there can be the problem of lowered polymerization activity of the resulting catalyst or the stereoregularity of the polymer. After finishing the reaction, the mixture in slurry state may be contacted with a titanium halide compound for the third time, and then washed with an organic solvent and dried to finally produce a catalyst for olefin polymerization.

The catalyst for olefin polymerization produced by the above described method comprises magnesium, titanium, electron donor compound and halogen atom, and the content of each component varies depending on the specific production procedure, but preferably contains 20-30% by weight of magnesium, 1-10% by weight of titanium, 5-20% by weight of electron donor compound, and 40-70% by weight of halogen atom.

The method of polymerizing olefin according to the present invention features in using the catalyst for olefin polymerization produced by the above described method, an alkyl aluminum and an external electron donor.

Any olefin can be used in the above method as long as the olefin is conventionally used in general olefin polymerization process, but propylene is preferable.

The above method of polymerizing olefin can be carried out by using the above components through slurry polymerization, bulk polymerization and gas state polymerization.

Among the components, the alkyl aluminum is a compound represented by the general formula AlR¹₃, where R¹ is a C₁₋₄ alkyl group, and specific examples include trimethylaluminum, triethylaluminum, tripropylaluminum, tributhylaluminum and triisobuthylaluminum.

Among the above components, the external electron donor is a compound represented by the general formula R²ₘSi(OR³)₄₋ₘ, where R² is an alkyl group or a cycloalkyl group of C₁₋₁₀, R³ is a C₁₋₃ alkyl group, and m is 1 or 2, and when m is 2, the two R² can be the same or different. The specific examples of the external electron donor include n-C₃H₇Si(OCH₃)₃, (n-C₃H₇)₂Si(OCH₃)₂, i-C₃H₇Si(OCH₃)₃, (i-C₃H₇)₂Si(OCH₃)₂, n-C₄H₉Si(OCH₃)₃, (n-C₄H₉)₂Si(OCH₃)₂, i-C₄H₉Si(OCH₃)₃, (i-C₄H₉)₂Si(OCH₃)₂, t-C₄H₉Si(OCH₃)₃, (t-C₄H₉)₂Si(OCH₃)₂, n-C₅H₁₁Si(OCH₃)₃, (n-C₅H₁₁)₂Si(OCH₃)₂, (cyclopentyl)Si(OCH₃)₃, (cyclopentyl)₂Si(OCH₃)₂, (cyclopentyl)(CH₃)Si(OCH₃)₂, (cyclopentyl)(C₂H₅)Si(OCH₃)₂, (cyclopentyl)(C₃H₇)Si(OCH₃)₂, (cyclohexyl)Si(OCH₃)₃, (cyclohexyl)₂Si(OCH₃)₂, (cyclohexyl)(CH₃)Si(OCH₃)₂, (cyclohexyl)(C₂H₅)Si(OCH₃)₂, (cyclohexyl)(C₃H₇)Si(OCH₃)₂, (cycloheptyl)Si(OCH₃)₃, (cycloheptyl)₂Si(OCH₃)₂, (cycloheptyl)(CH₃)Si(OCH₃)₂, (cycloheptyl)(C₂H₅)Si(OCH₃)₂, (cycloheptyl)(C₃H₇)Si(OCH₃)₂, (phenyl)Si(OCH₃)₃, (phenyl)2Si(OCH₃)₂, n-C₃H₅Si(OC₂H₅)₃, (n-C₃H₅)₂Si(OC₂H₅)₂, i-C₃H₅Si(OC₂H₅)₃, (i-C₃H₇)₂Si(OC₂H₅)₂, n-C₄H₉Si(OC₂H₅)₃, (n-C₄H₉)₂Si(OC₂H₅)₂, i-C₄H₉Si(OC₂H₅)₃, (i-C₄H₉)₂Si(OC₂H₅)₂, t-C₄H₉Si(OC₂H₅)₃, (t-C₄H₉)₂Si(OC₂H₅)₂, n-C₅H₁₁Si(OC₂H₅)₃, (n-C₅H₁₁)₂Si(OC₂H₅)₂, (cyclopentyl)Si(OC₂H₅)₃, (cyclopentyl)₂Si(OC₂H₅)₂, (cyclopentyl)(CH₃)Si(OC₂H₅)₂, (cyclopentyl)(C₂H₅)Si(OC₂H₅)₂, (cyclopentyl)(C₃H₇)Si(OC₂H₅)₂, (cyclohexyl)Si(OC₂H₅)₃, (cyclohexyl)₂Si(OC₂H₅)₂, (cyclohexyl)(CH₃)Si(OC₂H₅)₂, (cyclohexyl)(C₂H₅)Si(OC₂H₅)₂, (cyclohexyl)(C₃H₇)Si(OC₂H₅)₂, (cycloheptyl)Si(OC₂H₅)₃, (cycloheptyl)₂Si(OC₂H₅)₂, (cycloheptyl)(CH₃)Si(OC₂H₅)₂)₂, (cycloheptyl)(C₂H₅)Si(OC₂H₅)₂, (cycloheptyl)(C₃H₇)Si(OC₂H₅)₂, (phenyl)Si(OC₂H₅)₃, (phenyl)₂Si(OC₂H₅)₂ and the like.

In polymerizing olefin, appropriate portion of the cocotalyst, alkylaluminum, to the above described catalyst varies according to polymerization methods, but is 1-1,000 mole, or preferably 10-300 mole of aluminum atom in the cocatalyst to 1 mole of the titanium atom in the catalyst. When the portion of the alkylaluminum to the catalyst is outside the above range, there can be the problem that the polymerization activity of the catalyst significantly decreases.

In polymerizing olefin, appropriate portion of the external electron donor against the above described catalyst is 1-200 mole, or preferably 10-100 mole of silicon atom in the external donor to 1 mole of the titanium atom in the catalyst. When the portion of the external electron donor to the catalyst is outside the above range, there can be the problem that the polymerization activity significantly decreases.

### Advantageous Effect

According to the method of the present invention, it is possible to control the content of large particles in the produced dialkoxymagnesium support, and the particles have spherical shape. So, the catalyst produced by using the dialkoxymagnesium support of the present invention can have high activity, high stereoregularity and large bulk density, thereby making it possible to be applied to various commercial processes.

### EXAMPLES

The present invention will be described below in more detail with reference to the examples and comparative examples.

### Example 1

[Production of Spherical Support]

A 5 L glass reactor equipped with a stirrer, an oil heater and a reflux condenser was sufficiently ventilated by nitrogen, and 4.5 g of N-chlorosuccinimide, 60 g of magnesium metal (powdered product with average particle size of 100 μm) and 1000 ml of absolute ethanol were added to the reactor, and then stirrer was operated with 240 rpm at the reaction temperature of 60° C. After 10 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 60° C. After maintaining the reactor for 2 hours, the temperature was raised to 75° C. and aging was carried out at the temperature for 2 hours. After aging was completed, the resultant was washed 3 times at 50° C., using 2,000 ml of normal hexane each time. The washed resultant was dried for 24 hours under flowing nitrogen, and then 262 g of solid product (yield of 93.3%) in the form of white powder having good flowability was obtained. The average particle size of the dried product was 17.8 μm and the content of large particles of size not less than 75 μm was 4.6% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.8 μm, particle distribution index of 0.80 and bulk density of 0.29 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.12% by weight was obtained. The average particle size of the catalyst component was 18.2 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Example 2

[Production of Spherical Support]

A 5 L glass reactor equipped with a stirrer, an oil heater and a reflux condenser was sufficiently ventilated by nitrogen, and 4.5 g of N-chlorosuccinimide, 60 g of magnesium metal (powdered product with average particle size of 100 μm) and 1000 ml of absolute ethanol were added to the reactor, and then stirrer was operated with 240 rpm at the reaction temperature of 50° C. After 10 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 50° C. After maintaining the reactor for 2 hours, the temperature was raised to 75° C. and aging was carried out at the temperature for 2 hours. After aging was completed, the resultant was washed 3 times at 50° C., using 2,000 ml of normal hexane each time. The washed resultant was dried for 24 hours under flowing nitrogen, and then 273 g of solid product (yield of 97.2%) in the form of white powder having good flowability was obtained. The average particle size of the dried product was 17.2 μm and the content of large particles of size not less than 75 μm was 4.3% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.2 μm, particle distribution index of 0.78 and bulk density of 0.30 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.26% by weight was obtained. The average particle size of the catalyst component was 17.7 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Example 3

[Production of Spherical Support]

A 5 L glass reactor equipped with a stirrer, an oil heater and a reflux condenser was sufficiently ventilated by nitrogen, and 4.5 g of N-chlorosuccinimide, 60 g of magnesium metal (powdered product with average particle size of 100 μm) and 1000 ml of absolute ethanol were added to the reactor, and then stirrer was operated with 240 rpm at the reaction temperature of 45° C. After 10 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 45° C. After maintaining the reactor for 2 hours, the temperature was raised to 75° C. and aging was carried out at the temperature for 2 hours. After aging was completed, the resultant was washed 3 times at 50° C., using 2,000 ml of normal hexane each time. The washed resultant was dried for 24 hours under flowing nitrogen, and then 265 g of solid product (yield of 94.4%) in the form of white powder having good flowability was obtained. The average particle size of the dried product was 17.7 μm and the content of large particles of size not less than 75 μm was 4.7% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.7 μm, particle distribution index of 0.79 and bulk density of 0.31 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.23% by weight was obtained. The average particle size of the catalyst component was 18.1 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Example 4

[Production of Spherical Support]

A 5 L glass reactor equipped with a stirrer, an oil heater and a reflux condenser was sufficiently ventilated by nitrogen, and 4.5 g of N-chlorosuccinimide, 60 g of magnesium metal (powdered product with average particle size of 100 μm) and 1000 ml of absolute ethanol were added to the reactor, and then stirrer was operated with 240 rpm at the reaction temperature of 40° C. After 10 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 40° C. After maintaining the reactor for 2 hours, the temperature was raised to 75° C. and aging was carried out at the temperature for 2 hours. After aging was completed, the resultant was washed 3 times at 50° C., using 2,000 ml of normal hexane each time. The washed resultant was dried for 24 hours under flowing nitrogen, and then 277 g of solid product (yield of 98.3%) in the form of white powder having good flowability was obtained. The average particle size of the dried product was 16.8 μm and the content of large particles of size not less than 75 μm was 3.6% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 16.8 μm, particle distribution index of 0.76 and bulk density of 0.30 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.17% by weight was obtained. The average particle size of the catalyst component was 17.3 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Comparative Example 1

[Production of Spherical Support]

A 5 L glass reactor equipped with a stirrer, an oil heater and a reflux condenser was sufficiently ventilated by nitrogen, and 4.5 g of N-chlorosuccinimide, 60 g of magnesium metal (powdered product with average particle size of 100 μm) and 1000 ml of absolute ethanol were added to the reactor, and then stirrer was operated with 240 rpm at the reaction temperature of 75° C. for reflux state. After 5 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 75° C. for reflux state (aging process). After aging was completed, the resultant was washed 3 times at 50° C., using 2,000 ml of normal hexane each time. The washed resultant was dried for 24 hours under flowing nitrogen, and then 264 g of solid product (yield of 94.0%) in the form of white powder having good flowability was obtained. The average particle size of the dried product was 17.5 μm and the content of large particles of size not less than 75 μm was 25.4% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.5 μm, particle distribution index of 0.81 and bulk density of 0.31 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.17% by weight was obtained. The average particle size of the catalyst component was 17.8 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Comparative Example 2

[Production of Spherical Support]

A 5 L glass reactor equipped with a stirrer, an oil heater and a reflux condenser was sufficiently ventilated by nitrogen, and 5.5 g of N-bromosuccinimide, 60 g of magnesium metal (powdered product with average particle size of 100 μm) and 1000 ml of absolute ethanol were added to the reactor, and then stirrer was operated with 240 rpm at the reaction temperature of 75° C. for reflux state. After 5 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 75° C. for reflux state(aging process). After aging was completed, the resultant was washed 3 times at 50° C., using 2,000 ml of normal hexane each time. The washed resultant was dried for 24 hours under flowing nitrogen, and then 264 g of solid product (yield of 94.0%) in the form of white powder having good flowability was obtained. The average particle size of the dried product was 17.1 μm and the content of large particles of size not less than 75 μm was 47.5% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.1 μm, particle distribution index of 0.81 and bulk density of 0.31 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.10% by weight was obtained. The average particle size of the catalyst component was 17.6 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

### Comparative Example 3

[Production of Spherical Support]

A 5 L glass reactor equipped with a stirrer, an oil heater and a reflux condenser was sufficiently ventilated by nitrogen, and 5.5 g of N-bromosuccinimide, 60 g of magnesium metal (powdered product with average particle size of 100 μm) and 1000 ml of absolute ethanol were added to the reactor, and then stirrer was operated with 240 rpm at the reaction temperature of 50° C. After 10 minutes, as the reaction started and hydrogen is generated, the exit of the reactor was kept open so that the hydrogen gas was discharged, and the reactor was maintained at atmospheric pressure. After the generation of hydrogen was ceased, the reactor was further maintained for 2 hours at 50° C. Then the temperature was raised to 75° C. for reflux state, and stirred for 2 hours. After aging was completed, the resultant was washed 3 times at 50° C., using 2,000 ml of normal hexane each time. The washed resultant was dried for 24 hours under flowing nitrogen, and then 270 g of solid product (yield of 96.0%) in the form of white powder having good flowability was obtained. The average particle size of the dried product was 17.7 μm and the content of large particles of size not less than 75 μm was 38.1% by weight, which were measured by laser particle analyzer (Mastersizer X from Malvern Instruments) using light transmission method.

[Production of Solid Catalyst Component]

In a glass reactor equipped with a 1 L stirrer, which is sufficiently substituted with nitrogen, 150 ml of toluene and 25 g of the above prepared diethoxymagnisum with spherical shape having average particle size of 17.7 μm, particle distribution index of 0.83 and bulk density of 0.30 g/cc were added and maintained at 10° C. 25 ml of titanium tetrachloride diluted in 50 ml of toluene was added over 1 hour, and the temperature of the reactor was raised to 60° C. at a rate of 0.5° C. per minute. The reaction mixture was maintained for 1 hour at 60° C., then stirring was stopped and maintained until solid product was precipitated. After solid product was precipitated, supernatant liquid was removed, stirring was carried out for 15 minutes using 200 ml of toluene, and the resultant was washed once by the same method.

150 ml of toluene was added to the above solid product which was treated with titanium tetrachloride, and 50 ml of titanium tetrachloride was added at a constant rate over 1 hour while stirring with 250 rpm at 30° C. After completing the addition of titanium tetrachloride, 2.5 ml of diisobutylphtalate was added and the temperature of the reactor was raised to 110° C. at a constant rate (1° C./minute) over 80 minutes. While raising the temperature, 2.5 ml of diisobutylphtalate was further added at the moment when the temperature of the reactor reached 40° C. and 60° C. respectively. The temperature of the reactor was maintained at 110° C. for 1 hour, then lowered to 90° C. and stirring was stopped. Then supernatant was removed and the resultant was further washed 1 time using 200 ml of toluene with the same method. Then 150 ml of toluene and 50 ml of titanium tetrachloride were added and the temperature was raised to 110° C. and the system was maintained at the temperature for 1 hour. After completing aging process, the slurry mixture was washed 2 times using 200 ml of toluene each time, and then 5 times using 200 ml of normal hexane each time at 40° C., yielding solid catalyst component of light yellow color. By drying the component under flowing nitrogen for 18 hours, solid catalyst component with titanium content of 2.10% by weight was obtained. The average particle size of the catalyst component was 18.1 μm, which was measured by laser particle analyzer (Mastersizer X from Malvern Instruments) using light transmission method on the solid catalyst suspended in normal hexane.

[Polymerization of Propylene]

A small glass tube filled with 5 mg of the above prepared catalyst was installed in the high pressure stainless steel reactor of capacity of 2 L, and the reactor was sufficiently substituted with nitrogen. 3 mmol of triethylaluminum was added along with 0.15 mmol of cyclohexyl-methyldimethoxysilane (here, cyclohexyl-methyldimethoxysilane was used as an external electron donor). Then, 1000 ml of hydrogen and 1.2 L of liquid state propylene were added one after another, and after raising the temperature to 70° C., the stirrer was operated so that the glass tube installed in the reactor was broken and polymerization started. One hour after the start of polymerization, the temperature of the reactor was lowered to the ambient temperature and the propylene inside the reactor was completely degased by opening a valve.

Table 1 shows the content of large particles in the spherical support obtained by the examples 1-4 and comparative examples 1-3, the catalyst activity and the bulk density of the polymer.

The catalyst activity and the bulk density (BD) are calculated as follows:

Catalyst activity (kg-PP/g-cat)=the amount of polymer produced (kg)/the amount of catalyst (g)

Bulk density (BD)=the value measured according to ASTM D1895

TABLE 1

| | | | Initial | | |
|---|---|---|---|---|---|
| | | Content of | reaction | Activity | Bulk |
| | | large molecule | temperature | (kg-PP/ | density |
| | Initiator | (% by weight) | (° C.) | g-cat) | (BD) |
| Example 1 | NCS | 4.6 | 60 | 55.4 | 0.46 |
| Example 2 | NCS | 4.3 | 50 | 57.3 | 0.45 |
| Example 3 | NCS | 4.7 | 45 | 55.8 | 0.46 |
| Example 4 | NCS | 3.6 | 40 | 54.7 | 0.46 |
| Comparative | NCS | 25.4 | 75 | 52.1 | 0.45 |
| Example 1 | | | | | |
| Comparative | NBS | 47.5 | 75 | 53.5 | 0.45 |
| Example 2 | | | | | |
| Comparative | NBS | 38.1 | 50 | 55.1 | 0.44 |
| Example 3 | | | | | |

NCS: N-Chlorosuccinimide,
NBS: N-bromosuccinimide
Large pacticle: particle with size equal to or largen than 75 μm

As can be seen in Table 1, less than 5% by weight of large particles were produced in the Examples 1-4, where NCS was used as an initiator and the reaction was carried out at lowered initial reaction temperature of 40-60° C., which is significantly lower than the result of the Comparative Example 1, where the reaction was carried out at reaction temperature of 75° C. Also, in the Comparative Example 3, where the reaction was carried out at lowered reaction temperature, but NBS was used as an initiator, more than 30% by weight of large particles were produced showing that the initiator affected the formation of large particles. Therefore, by using the solid catalyst component, which is produced by using the support prepared at low temperatures using NCS as in Examples 1-4, along with the mixture of alkylaluminum and an external electron donor in the olefin polymerization, the catalyst activity is the same or higher compared to the conventional catalyst component and olefin polymer having improved bulk density, which greatly affects the productivity of commercial manufacturing, can be produced with high yield.

1. A method for producing a dialkoxymagnesium support for catalyst for olefin polymerization by reacting a magnesium metal with an alcohol under the presence of an initiator, wherein the initiator is N-chlorosuccinimide, and an initial reaction temperature is 40-60° C.

2. The method for producing a dialkoxymagnesium support for catalyst for olefin polymerization of claim 1, wherein the amount of the initiator used is 0.001-0.2 parts by weight per 1 part by weight of the magnesium metal.

3. A method for producing a catalyst for olefin polymerization comprising contact-reacting the dialkoxymagnesium support produced by the method of claim 1 with a titanium halide compound and an internal electron donor.

4. A method for polymerizing olefin comprising polymerizing olefins in the presence of the catalyst for olefin polymerization produced by the method of claim 3, an alkyl aluminum and an external electron donor.

5. A method for producing a catalyst for olefin polymerization comprising contact-reacting the dialkoxymagnesium support produced by the method of claim 2 with a titanium halide compound and an internal electron donor.

6. A method for polymerizing olefin comprising polymerizing olefins in the presence of the catalyst for olefin polymerization produced by the method of claim 5, an alkyl aluminum and an external electron donor.
